(19) United States
(12) Patent Application Publication
(10) Pub. No.: US 2010/0256672 A1
(43) Pub. Date: Oct. 7, 2010

(54) APPARATUS AND METHOD FOR WOUND WEAVING AND HEALING

(75) Inventors: Irving Weinberg, Bethesda, MD (US); Mario Urdaneta, Berwyn Heights, MD (US); Pavel Stepanov, North Potomac, MD (US); Elisabeth Smela, Silver Spring, MD (US)

Correspondence Address: BARNES & THORNBURG LLP, 750-17TH STREET NW, SUITE 900, WASHINGTON, DC, 20006-4675, US

(73) Assignee: WEINBERG MEDICAL PHYSICS LLC, Bethesda, MD (US)

(21) Appl. No.: 12/750,034

(22) Filed: Mar. 30, 2010

Related U.S. Application Data
(60) Provisional application No. 61/165,745, filed on Apr. 1, 2009.

(51) Int. Cl. A61B 17/03

(52) U.S. Cl. 606/214

(57) ABSTRACT

A method and apparatus for closing a gap between tissues, comprising introducing a device head into the gap, applying a force to a portion of one tissue with the device head, applying a force to a portion of the other tissue with the device head, moving, via the device head, the first tissue into close proximity to the second tissue, maintaining, for a predetermined period of time via the device head, the one tissue and the other tissue in close proximity until the portion of the one tissue and the portion of the other tissue become cellularly adhered to one another, thereby forming a cellular aggregate connecting the tissues, and releasing from the device head the portion of the one tissue, the portion of the other tissue, and the cellular aggregate after a predetermined period of time of less than about one minute.

### CROSS-REFERENCE TO RELATED APPLICATION(S)

This application claims the benefit of U.S. Provisional Application No. 61/165,745 filed Apr. 1, 2009, the contents of which are incorporated herein by reference.

### FIELD OF THE INVENTION

The present invention concerns an apparatus and a method for wound weaving and healing where cells at a wound's edges are reattached to one another via manipulation of the tissue.

### BACKGROUND OF THE INVENTION

Traditionally methods for wound healing rely on the body's intrinsic tissue regeneration processes.

Surgery, for example, relies on the body's intrinsic healing processes, which employ fibroblasts, microphages, and granulation tissue to effectuate healing of a specific wound. Some have suggested that this natural process, which evolved to heal bite wounds and major traumas, is poorly suited for the repair of the types of fine cuts delivered by surgeons.

With the advent of microsurgery, it has become possible to more accurately align macroscopic structures under repair by making even finer cuts in the tissue. However, even these advancements have not altered the basic approach to reliance on cellular processes for wound repair.

Cellular processes during wound repair may be summarized as follows.

The first wound healing phase is referred to as the inflammatory phase. In this phase, injured blood vessels promote the formation of a fibrin plug and the formation of platelet aggregation. The released platelets elaborate substances that attract white blood cells and fibroblasts. White blood cells debride the wound and release substances that further attract fibroblasts and epithelial cells. In other words, white blood cells remove contaminants from the tissue, as should be appreciated by those skilled in the art.

The second wound healing phase is referred to as the proliferative phase. In the proliferative phase, fibroblasts migrate to the wound center over a previously-established fibrin lattice. The fibroblasts start producing components of granulation tissue and collagen. In this second phase, intact vessels expand their reach into the wound.

The third and final phase of wound healing is referred to as the maturation phase. During the maturation phase, collagen is deposited into the wound and matured, aligning along the lines of tension in the wound. Epithelial cell progenitors (e.g., keratinocytes) migrate from the wound edges until the defect is covered. As the epithelial cells climb over one another in the middle of the wound, they dissolve the clot and the old basement membrane. When the epithelial cells meet, they stop their migration (i.e., contact inhibition) and apply desmosomes to anchor themselves to each other and to the basement membrane. In this phase, some fibroblasts become myofibroblasts that contract the wound, thereby promoting wound closure.

As should be appreciated by those skilled in the art, there are potential problems and difficulties associated with the natural wound-healing process.

For example, systemic and genetic maladies may negatively affect wound healing.

In addition, patients with impaired peripheral circulation (e.g., smokers) cannot deliver enough oxygen to cells required for wound healing. This oxygen supply defect is further aggravated in diabetics, due to impaired phagocytosis from abnormal glucose concentrations.

Also, it is known that hypertrophy of scar tissue occurs in a significant portion of patients after surgery. This is referred to as hypertrophic scarring.

In many African and Asian patients, keloids may form, in which the scar exceeds the size of the original wound. Unlike hypertrophic scarring, keloid lesions generally do not respond to subsequent attempts at surgical repair.

Next, it is known that healing problems with internal wounds, as are common with abdominal or pelvic surgery, may cause adhesions, which lead to significant morbidity.

Experimental studies indicate that hypertrophy occurs as a result of signaling errors between various cellular components involved in the wound-healing process.

In addition to local and circulating humoral factors, mechanical factors may affect signaling between cellular components of the wound-healing process. For example, when keratinocytes are separated from fibroblasts, collagen synthesis increases. Runaway collagen synthesis is believed to be the cause of keloids and scar hypertrophy.

It is known that surgical “best practices” may improve post-surgical appearances include judicious selection of layered sutures with minimal perturbation of vascular supply and overall tissue integrity.

The local application of chemical and biological factors has been proposed, in order to alter growth and signaling processes. These topically-applied remedies include stem cells and growth factors that promote stem-cell like behavior.

Application of vacuum to wounds has been studied (vacuum-assisted closure), with generally favorable results. (See, e.g., L. C. Argenta and M. J. Morykwas, “Vacuum-assisted closure: a new method for wound control and treatment-clinical experience,” Annals of Plastic Surgery, Volume 38, pages 563-576 (1997)).

Vacuum application is believed to reduce edema, increase production of granulation tissue, and improved orientation of cells with respect to one another.

Additionally, it is noted that silicone gel sheets have been shown to improve wound healing, possibly because of reduced wound deformation during patient motion.

In addition to the foregoing, manufacturers of certain surgical knives and cautery systems have described improved scar appearances when their proprietary implements are used, e.g., plasma blades.

In addition, microsurgical techniques can be improved with robotic techniques that reduce hand tremor, and coupling devices that improve vascular patency.

As should be apparent from the foregoing, there are a number of disparate factors that may hinder or negatively impact wound healing when reliance is placed on the body's natural healing processes.

As also should be apparent from the foregoing, a person's body may not effectuate wound healing in a manner considered ideal by surgeons, doctors, and other medical practitioners. This includes scarring, which is of particular concern with respect to cosmetic surgery.

As a result, there has developed a need for methods and devices that may facilitate wound healing, among others. This includes methods and devices that improve upon the body's natural wound-healing processes.

In addition, there has developed a need for methods and devices that promote wound healing to minimize scarring, among others.

### BRIEF SUMMARY OF THE INVENTION

It is an object of the invention to provide a method for closing a gap between tissues, comprising: introducing a device head into the gap between a first tissue and a second tissue; applying a force to a portion of the first tissue with the device head; applying a force to a portion of the second tissue with the device head; moving, via the device head, the portion of the first tissue into close proximity to the portion of the second tissue; maintaining, for a predetermined period of time via the device head, the portion of the first tissue and the portion of the second tissue in close proximity until the portion of the first tissue and the portion of the second tissue become cellularly adhered to one another, thereby forming a cellular aggregate that connects the portion of the first tissue to the portion of the second tissue; and releasing from the device head the portion of the first tissue, the portion of the second tissue, and the cellular aggregate after the predetermined period of time; wherein the predetermined portion of time is less than about one minute.

It is another object of the invention to provide An apparatus for closing a gap between tissues, comprising a device head for insertion in a gap between a first tissue and a second tissue, wherein the device head comprises a first side region, a second side region disposed opposite to the first side region, a leading edge, and a trailing edge disposed opposite to the leading edge; a first plurality of openings disposed through the first side region; a second plurality of openings disposed through the second side region; and a suction generator connected to the first plurality of openings; wherein application of suction by the suction generator to the first plurality of openings is adapted to pull a portion of the first tissue into close proximity with a portion of the second tissue so that as the device head is moved in the gap the suction permits the portion of the first tissue and the portion of the second tissue to contact and become adhered to one another, thereby forming a cellular aggregate connecting the portion of the first tissue to the portion of the second tissue.

Other aspects of the present invention will be made apparent from the in the discussion that follows.

### BRIEF DESCRIPTION OF THE DRAWINGS

The present invention will now be described in connection with one or more drawings, in which:

FIG. 1 is a schematic illustration of an open wound with a suction-based device according to the present invention being disposed adjacent thereto;

FIG. 2 is a schematic illustration of the suction-based device illustrated in FIG. 1 during the time that suction is applied to the wound edges;

FIG. 3 is a schematic illustration of the suction-based device illustrated in FIGS. 1 and 2, after the application of suction, showing the edges of the wound being adhered to one another;

FIG. 4 is a perspective illustration of the device head portion of a wound healing device according to the present invention;

FIG. 5 is a perspective illustration of the wound healing device illustrated in FIG. 4, showing various operations performed by the device;

FIG. 6 is a side view schematic of the wound healing device shown in FIGS. 4 and 5, the wound healing device being shown in a first position prior to travel of the device head through the identified area of the wound;

FIG. 7 is a cross-sectional view, schematic illustration of the condition of the wound at the position of the dotted line in FIG. 6, when the wound healing device is in the first position;

FIG. 8 is a side view schematic of the wound healing device shown in FIGS. 4 and 5, the wound healing device being shown in a second position where debris is being removed from the wound;

FIG. 9 is a cross-sectional view, schematic illustration of the condition of the wound at the position of the dotted line in FIG. 8, when the wound healing device is in the second position;

FIG. 10 is a side view schematic of the wound healing device shown in FIGS. 4 and 5, the wound healing device being shown in a third position where the wound edges are beginning to be drawn together;

FIG. 11 is a cross-sectional view, schematic illustration of the condition of the wound at the position of the dotted line in FIG. 10, when the wound healing device is in the third position;

FIG. 12 is a side view schematic of the wound healing device shown in FIGS. 4 and 5, the wound healing device being shown in a fourth position where the wound edges are partially reattached to one another;

FIG. 13 is a cross-sectional view, schematic illustration of the condition of the wound at the position of the dotted line in FIG. 12, when the wound healing device is in the fourth position;

FIG. 14 is a cross-sectional view, schematic illustration of a wound prior to a first pass of the wound healing device of the present invention;

FIG. 15 is a cross-sectional view, schematic illustration of a wound after a first pass of the wound healing device of the present invention;

FIG. 16 is a cross-sectional view, schematic illustration of a wound after a second pass of the wound healing device of the present invention;

FIG. 17 is a cross-sectional view, schematic illustration of a wound after an n^{th} pass of the wound healing device of the present invention;

FIG. 18 is a side view of one contemplated embodiment of the wound healing device of the present invention;

FIG. 19 is an enlarged side view of the wound healing device shown in FIG. 18;

FIG. 20 is a depiction of a photograph of a tissue sample, showing the adhesion of the two parts of the tissue after application of the wound healing device of the present invention;

FIG. 21 is a depiction of a photograph of a tissue sample, showing the lack of adhesion of the two parts of the tissue in the case where the wound healing device of the present invention is not applied;

FIG. 22 is a first part of a flow diagram illustrating one method contemplated by the present invention; and

FIG. 23 is a second part of the flow diagram illustrating one method contemplated by the present invention.

### DETAILED DESCRIPTION OF THE INVENTION

The present invention will now be described in connection with one or more contemplated embodiments. The embodiments discussed are not intended to be limiting of the scope of the present invention. To the contrary, the embodiments described herein are intended to be exemplary of the broad scope of the present invention. In addition, those skilled in the art will appreciate certain variations and equivalents of the embodiments described herein. The present invention is intended to encompass those equivalents and variations as well.

One aspect of the present invention is to provide an apparatus and a method that promotes wound healing. Another aspect is to provide an apparatus and a method that assists the body's natural wound-healing processes to expedite wound healing. A further aspect is to provide an apparatus and a method that helps to effectuate improved wound healing by comparison with unassisted wound healing.

In one embodiment, for example, rows of cells at the wound edges are microscopically re-attached to one another (e.g., the cells are “zippered” together) using a hand-held vacuum-assisted intraoperative device. This cellular re-attachment process uses micro-mechanical manipulation techniques similar to those employed in micro-dissection, in order to achieve wound repair with minimal cellular perturbation.

Based at least upon pilot experimental data, it is predicted that the apparatus and the method of the present invention may reduce the roles of fibroblasts and granulation tissue in wound-healing processes. By taking advantage of intrinsic desmosome-mediated attraction mechanisms between cells, it is possible to lower the amount of suture materials required to effectively close wounds, thereby reducing the chance of infection.

An object of the present invention is to reduce or eliminate the need for sutures, staples, adhesives, and other tissue connecting devices and substances that are traditionally employed, especially during surgical procedures. As a result of the application of the apparatus and/or method of the present invention there is a reduction in the amount of scarring in plastic surgery, with long-term applications to other healing processes. It is contemplated to replace the natural process of cellular migrations with tissue engineering at the cellular level, in which dangling cells at the wound edges are configured into optimal orientations with respect to one another. The present invention makes use of micro-manipulation and advanced micro-and meso-fabrication techniques.

In one embodiment of the present invention, a vacuum device, such as a suction device, is relied upon to provide the mechanism by which wound healing is facilitated. One of the benefits of vacuum-assisted wound closure is the tendency for negative pressure to draw the edges of a wound closer to each other.

The present invention relies, at least in part, on the hypothesis that, by coherently re-attaching cells to one another near the wound edge, it is possible (with minimal perturbation) to reduce cell separation (e.g., of keratinocytes from fibroblasts), to more correctly align cells, and to minimize the chances of exuberant collagen synthesis. These effects may increase tissue strength, while reducing the need for suture materials. This reduction in the use of suture materials, which sometimes act as foreign bodies in the wound, potentially decreases the likelihood of infection from micro-organisms.

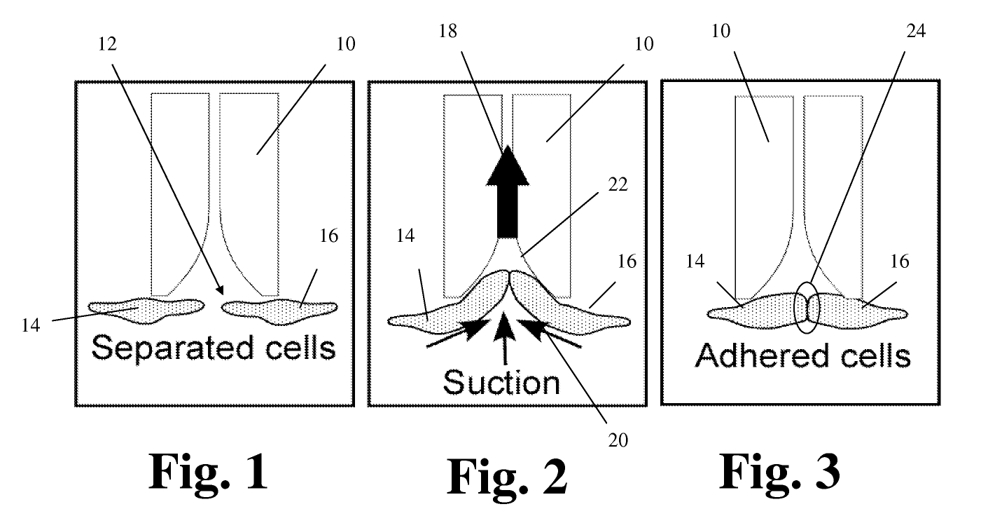

FIGS. 1-3 are schematic illustrations detailing the approach of the present invention. These figures are based on preliminary studies of tissue adherence in accordance with the present invention. Those preliminary studies demonstrated that controlled suction and pulsion with shaped nozzles may be used to re-attach cells to one another. These studies showed that a “bridge” may be constructed across a wound by manipulating the tissues at either edge of a wound into close proximity to one another.

In the preliminary studies, a suction device, herein referred to as a wound healing device 10 (also referred to herein as a “device” and/or a “tool”) was placed into close proximity to a wound 12. The wound 12 is shown in FIG. 1 as having two halves, a first tissue portion 14 and a second tissue portion 16. As illustrated in FIG. 1, the first and second tissue portions 14, 16 are separated from one another. This is to be expected for any type of wound 12.

As a preliminary matter, it is anticipated that the first and second tissue types 14, 16 are of the same type. For example, in a wound 12 in a person's skin, the first and second tissue portions 14, 16 may be skin (i.e., epidermal or dermal) cells. However, it should be noted that the two tissue portions 14, 16 need not be of the same type. In other words, the types of cells that make up the first and second tissue portions 14, 16 need not be of the same type to practice the present invention.

With continued reference to FIG. 1, the wound healing device 10 is placed into close proximity to the wound 12, adjacent to the separation between the first and second tissue types 14, 16. In FIG. 1, the wound healing device 10 has not yet been activated.

It is contemplated that the wound healing device 10 is a suction device. The wound healing device 10 may be in the form of a pipette. As should be apparent to those skilled in the art and as discussed in greater detail herein, the wound healing device 10 may take any of alternative constructions. A pipette is but one example.

FIG. 2 illustrates the condition of the wound 12 after the wound healing device is activated. In this illustration, and consistent with the approach where the wound healing device applies suction, suction 18 is being applied to the first and second tissue types 14, 16. As a result of applying suction 18, forces 20 are exerted on the first and/or second tissue portions 14, 16 to draw them toward the suction inlet 22 of the wound healing device 10.

After the first and/or second tissue portions 14, 16 are drawn to the suction inlet 22, suction 18 is maintained for a predetermined period of time to permit the cells in the first and second tissue portions 14, 16 to adhere to one another via cellular adherence. After the predetermined period of time, the suction 18 is discontinued.

FIG. 3 illustrates the status of the first and second tissue types 14, 16 after the suction 18 is discontinued. The cells that make up the first and second tissue portions 14, 16 will have formed a cellular aggregate 24. The cellular aggregate 24 is the bridge between the first and second tissue types 14, 16.

In the stage illustrated in FIG. 3, the suction 18 has been discontinued. As a result, the cellular aggregate 24 has been released. As a general rule, it is anticipated that cessation of the suction 18 will result in the release of the cellular aggregate 24. However, it is anticipated that there may be instances where pressure (i.e., reverse suction or pulsion) may need to be applied to release the first and second tissue portions 14, 16 and the cellular aggregate 24 from the suction inlet 22.

As may be appreciated from FIGS. 1-3, the wound healing device 10 is analogous to a “spot welder.” Specifically, the suction inlet 22 establishes a cellular aggregate 24 for the spot where the first and second tissue portions 14, 16 are drawn together. Once the suction 18 is released, the region where suction 18 has been applied will contain a cellular aggregate 24. By repeating the application of the wound healing device 10 repetitively, it is possible to close a wound 12. For example, each repetitive pass of the wound healing device 10, 28 may be at a more superficial level of the wound along the length of the wound until the depth of the wound is closed from bottom to top.

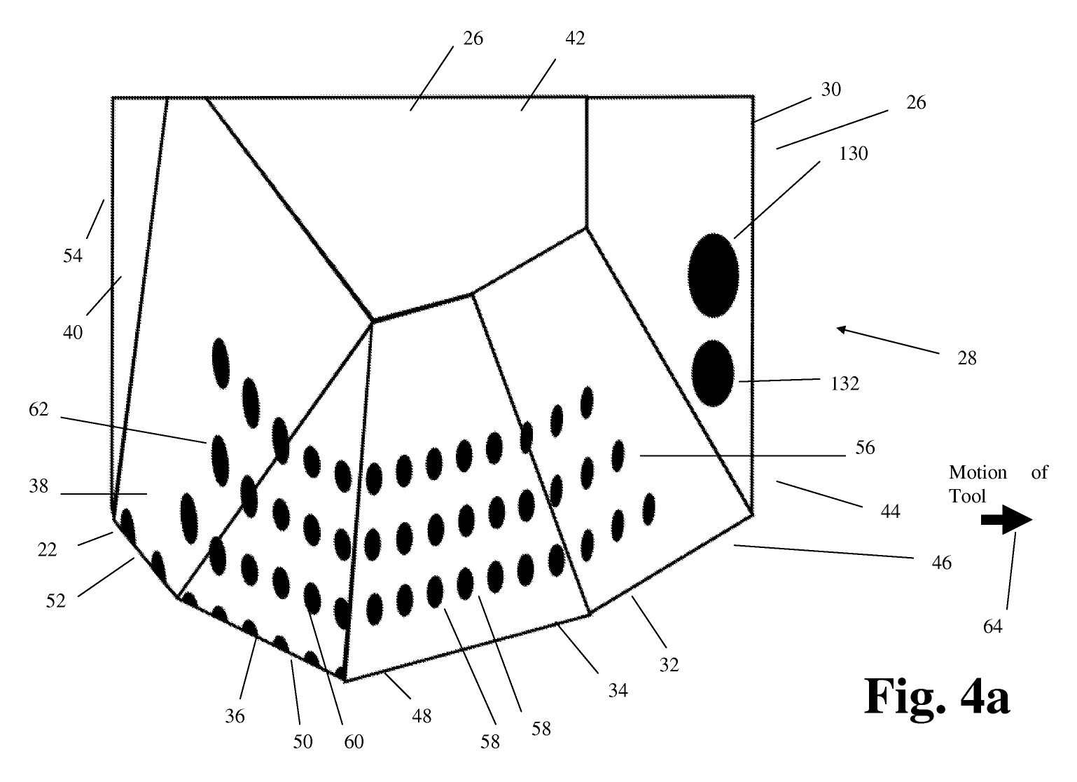

FIG. 4 is a close-up illustration of one embodiment of a device head 26 of a wound healing device 28 according to the present invention. This embodiment of the wound healing device is intended to function in a fashion to establish a continuous cellular aggregate 24 from one end of the wound 12 to the other.

The device head 26 includes on at least one side seven surfaces 30, 32, 34, 36, 38, 40, 42, some of which are perforated. Preferably, the device head 26 has the seven surfaces on each of two sides, for a total of fourteen surfaces per device head. The seven surfaces are: (1) the leading edge surface 30, (2) the first preparation edge surface 32, (3) the second preparation edge surface 34, (4) the first healing edge surface 36, (5) the second healing edge surface 38, (6) the trailing edge surface 40, and (7) the side surface 42. These seven surfaces are repeated on the side of the device head 26 of the wound healing device 28 that is on the side opposite to the view shown in, for example, FIG. 4.

The leading edge 44 is one of six edges: the other five are (1) the first preparation edge 46, (2) the second preparation edge 48, (3) the first healing edge 50, (4) the second healing edge 52, and (5) the trailing edge 54.

As should be immediately apparent, there are numerous variations and equivalents that are contemplated to be encompassed by the present invention. For example, a larger number or a smaller number of surfaces and/or edges may be employed without departing from the scope of the invention. In addition, the wound healing device may not incorporate distinct surfaces and/or edges. Instead, the wound healing device may be curved in appearance from its leading edge 44 to its trailing edge 54. In yet another contemplated embodiment, a combination of flat surfaces and curved surfaces (and/or edges) may makeup the wound healing device head without departing from the scope of the present invention.

As shown in FIG. 4, the leading edge surface 30 defines a leading edge 44 of the device head 26 of the wound healing device 28. The leading edge surface includes two perfusion holes 130, 132. Perfusion holes 130, 132 are located adjacent the leading edge 44.

The perfusion holes 130, 132 are provided so that liquids may be introduced into the wound 12 prior to formation of the cellular aggregate 24. In one contemplated embodiment, the perfusion holes 130, 132 may introduce fluids including anti-inflammatory and/or anti-infection agents. Alternatively, the perfusion holes 130, 132 may introduce a saline solution to assist with cleaning of the wound 12 prior to formation of the cellular aggregate 24. The perfusion holes 130, 132 also may be employed to introduce growth-enhancing or agglomerating factors such as steroids and/or other stimulants or adhesive materials. As may be appreciated by those skilled in the art, the number of perfusion holes and the different types of medicaments and fluids that may be discharged through the perfusion holes 130, 132 may vary.

The leading edge surface 30 is positioned adjacent to a first preparation edge 32. The first preparation edge 32 is provided with a plurality of apertures 56. The second preparation surface 34 includes a plurality of apertures 58. The first healing surface 36 includes several apertures 60. Finally, the second healing edge surface 38 also includes a plurality of apertures 62. As will be discussed in greater detail below, the apertures 56, 58, 60, 62 apply suction and/or pulsion to the first and second tissue portions 14, 16.

With respect to the device head 26, it is contemplated that the apertures 56, 58, 60, 62 will work in unison to permit the first and second tissue portions 14, 16 to be adhered together.

As shown in FIG. 4, the device head 26 of the wound healing device 28 is intended to move in a tool direction 64. This presents a challenge for the tool 28, which must hold the first and second tissue portions 14, 16 in close proximity for a predetermined period of time.

To assure cellular adhesion as the device head 26 of the wound healing device 28 moves in the tool direction 64, the apertures 56, 58, 60, 62 successively apply suction and/or pulsion so that the first and second tissue portions 14, 16 are successively handed from one set of apertures to the next set of apertures. As a result of this continual hand-off procedure between apertures and/or groups of apertures, the first and second tissue portions 14, 16 may be held together for a sufficient duration to permit the creation of a continuous cellular aggregation 24 in the wound 12.

As should be apparent to those skilled in the art, the apertures 56, 58, 60, 62 may be operated independently from one another. Alternatively, the apertures 56, 58, 60, 62 may be operated in groups. In one contemplated example, the apertures 56 may apply a suction 18 to the first and second tissue portions 14, 16. As the device 28 advances in the tool direction 64, the apertures 58 may be activated in unison or in sequence to provide suction and other apertures such as the apertures 56 may be placed into a non-suction (and/or pulsion) mode of operation. The hand-off may then be made from the apertures 58 to the apertures 60, and so on. Naturally, when the apertures 60 are in a suction mode, the apertures 56 also are anticipated to be in a suction mode. Similarly, when the apertures 58 are in a suction mode, the apertures 62 may also be in a suction mode. Other variations and equivalents of this methodology also are intended to fall within the scope of the invention.

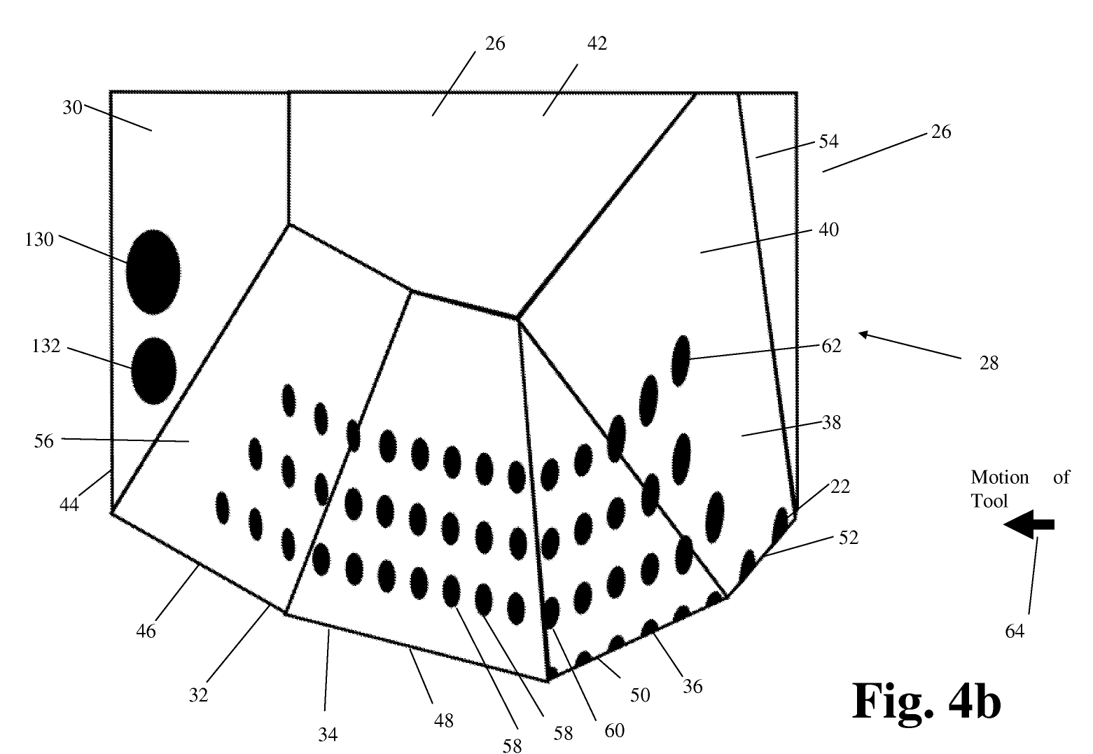

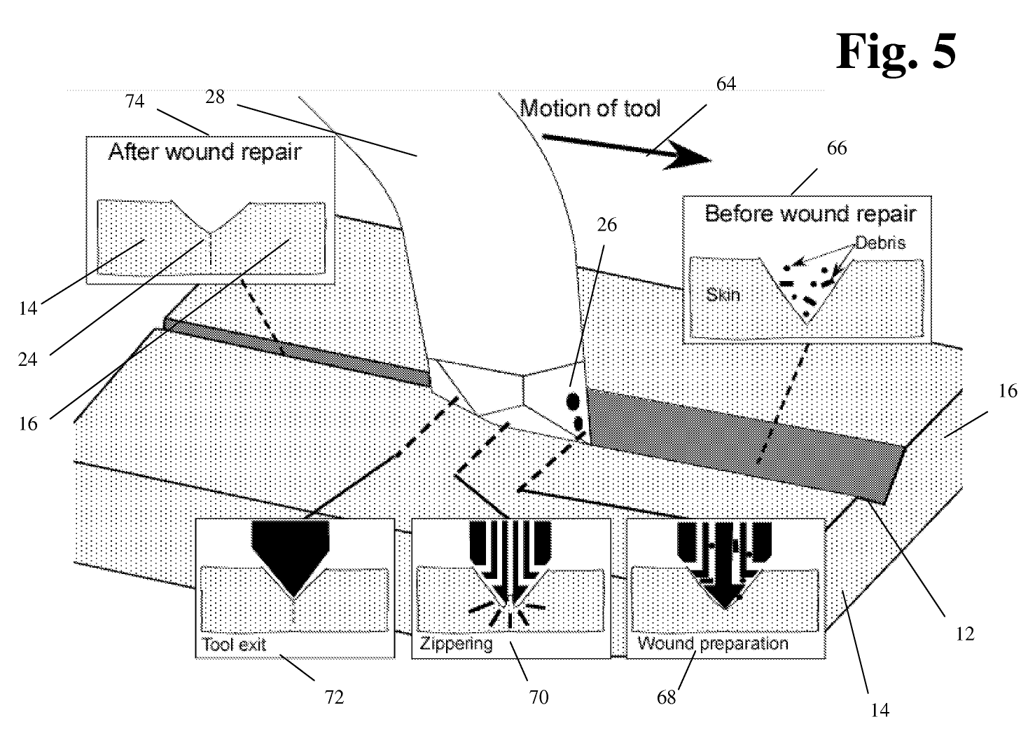

Reference is now made to FIG. 5, which provides a perspective illustration of the wound healing device 28 as shown in FIG. 4 in an operative mode.

The wound healing device 28 includes the device head 26 described above in connection with FIG. 4. The tool direction 64 is also illustrated, as shown in FIG. 4, as are the first and second tissue portions 14, 16 and the wound 12. FIG. 5 also provides illustrations of the various stages of wound healing that occur as the wound healing device 28 passes through the wound 12.

Initially, before the wound healing device 28 passes through the wound 12, the wound 12 is open, as shown in the insert labeled 66. The open wound 66 may include various materials, such as debris. The debris may be cleared from the open wound 66 via fluids injected into the open wound 66 through the perfusion holes 130, 132. As the wound healing device 28 passes through the wound 12, eventually, the device 28 reaches a wound preparation stage 68. At the wound preparation stage 68, the wound healing device 28 applies suction to remove the debris from the wound 12. It is also contemplated that the wound healing device 28 may abrade the side surface of the wound 12 to facilitate creation of the cellular aggregation 24. The wound healing device 28 then proceeds to the zippering stage 70 where suction is applied to pull the first and second tissue portions 14, 16 toward one another. After the zippering stage 70, the wound healing device 28 moves to an exit stage 72. At the wound exit stage 72, the suction 18 has been discontinued so that the tissue portions 14, 16 are released. The status of the wound 12 is illustrated in the repaired stage 74 after suction has been discontinued. As shown, the tissue portions 14, 16 have been adhered to one another and have formed the cellular aggregate 24.

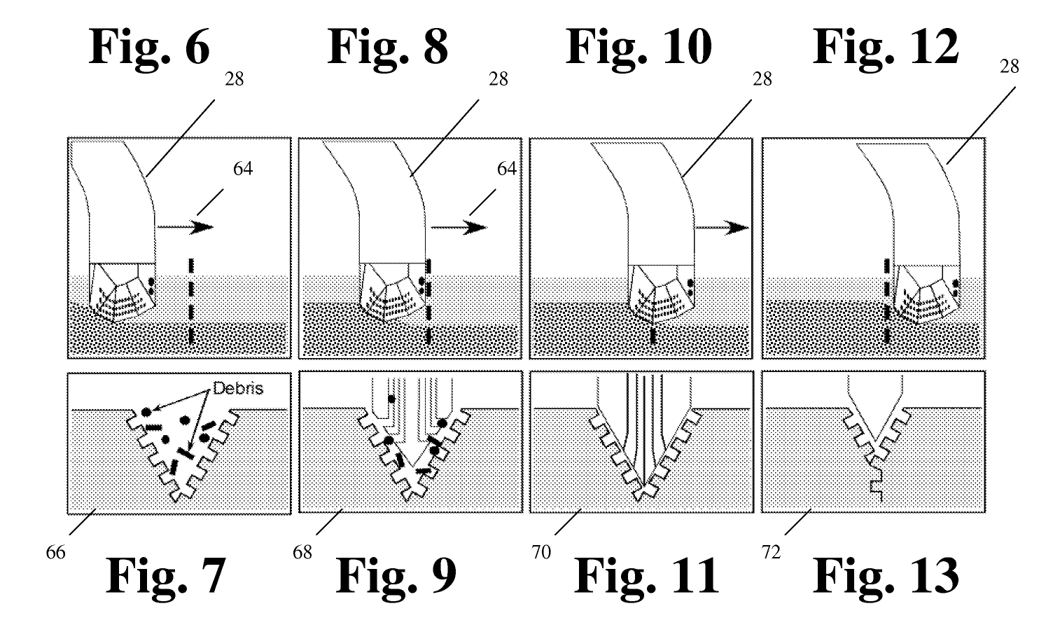

As should be immediately apparent from FIG. 5, and as will be described in greater detail below in connection with FIGS. 6-17, the wound 12 is not necessarily completely healed after the wound healing device 28 is passed therethrough. For example, as shown in FIG. 5, the wound repair stage 74 indicates that further healing may be desired or required. It is contemplated that the wound healing device 28 may be passed through the wound 12 in several iterations before the wound 12 is completely (or nearly completely) closed. Conversely, depending on the type of tissue, etc. one pass may be sufficient to provide wound healing.

FIGS. 6-13 are intended to be viewed as a group of figures that illustrate the various stages associated with the application of the wound healing device 28 to a wound 12.

FIG. 6 provides a side view of the wound healing device 28 in a wound 12. The dotted line indicates the position at which a cross-section is taken, which is illustrated in FIG. 7. In keeping with the discussion of FIG. 5, above, FIGS. 6 and 7 collectively illustrate the condition of the wound 12 in the open wound stage 66.

FIG. 8 provides a side view of the wound healing device 28 at a second position within the wound 12 along the travel direction 64. At this point, the wound healing device 68 is in the wound preparation stage 68. FIG. 9 is a cross-sectional illustration of the wound preparation stage, as illustrated in FIG. 8.

FIG. 10 provides a side view illustration of the wound healing device 28 in an advanced position with respect to the position shown in FIG. 8. In this illustration, the wound healing device 28 is in the zippering stage 70. A cross-section of the zippering stage 70 is provided in FIG. 11.

FIG. 12 is a side view of the wound healing device 28 in the exit stage 72, after zippering is complete. FIG. 13 is a cross-sectional side view of the exit stage 72.

In FIGS. 7, 9, 11, and 13, the sides of the wound 12 are shown with exaggerated, abraded surfaces. The wound healing device 28 may abrade the sides of the wound 12 in the wound preparation stage 66. It is contemplated that the wound healing device 28 may be provided with a scalpel or other suitable tool to create side walls to the wound 12 that may be more easily fused together as a result of cellular adhesion. For example, a scalpel 140 may protrude from surface 32 whereby tissue is severed to provide a desired shape of tissue surface to be joined. Scalpel 140 may also be configured to impart a desired roughness to the tissue whereby the scalpel 140 may be configured to abrade the tissue with or without cutting or shaping the tissue.

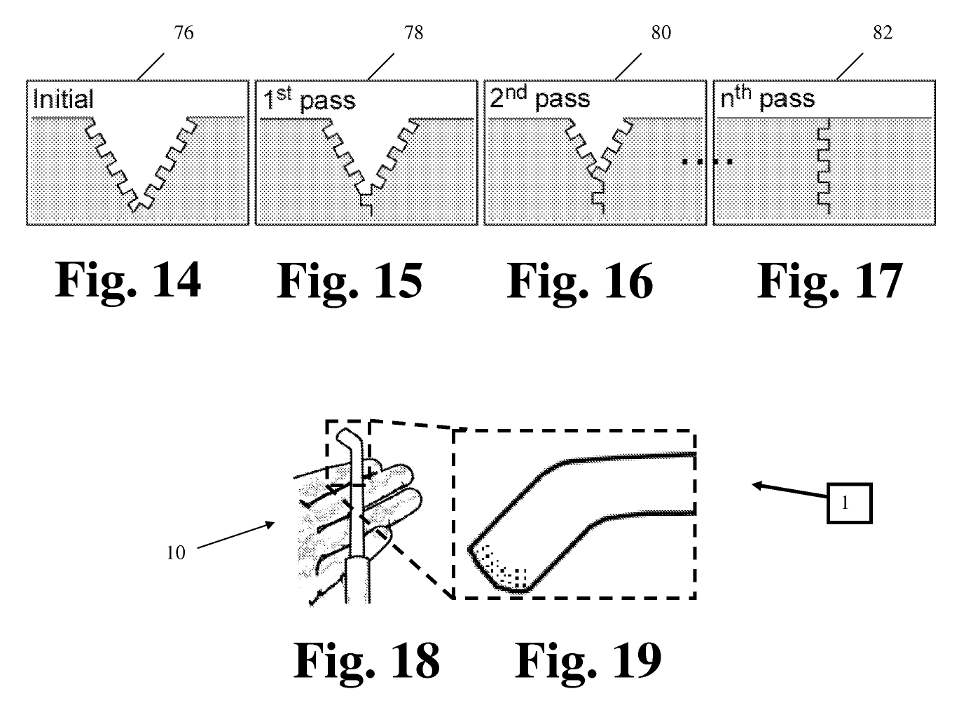

FIGS. 14 through 17 illustrate the stages contemplated when the wound healing device 28 of the present invention is applied repetitively to the wound 12.

FIG. 14 illustrates the wound 12 in an initial stage 76. In FIG. 15, after a first pass with the wound healing device, the wound 12 is shown in a first pass stage 78, where the bottom portion of the wound 12 has been cellularly adhered. In FIG. 16, the wound 12 is shown in a second pass stage 80, where the wound 12 nearly healed. When the wound 12 has been subjected to n passes of the wound healing device 28, the wound 12 will appear as in the n^{th} pass stage 82, which is illustrated in FIG. 17.

FIG. 18 provides a view of one contemplated construction for the wound healing device 10, discussed above. FIG. 19 is an enlarged view of the wound healing device 10.

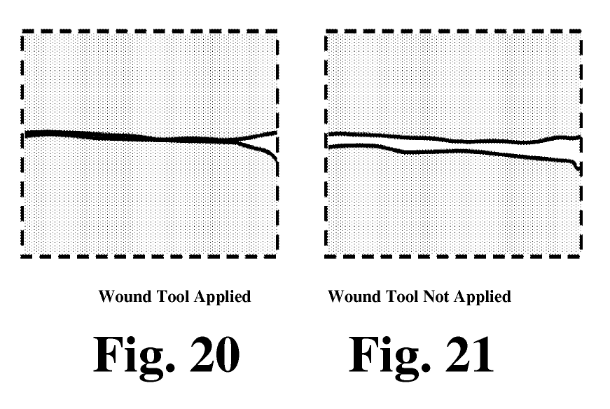

FIGS. 20 and 21 provide a comparative example of tissue where the wound healing device 10 has been applied versus tissue where the wound healing device 10 has not been applied. As is apparent from the comparison, where the wound healing device 10 is applied to the tissue, a cellular aggregation 24 forms to adhere the first and second tissue portions 14, 16 to one another.

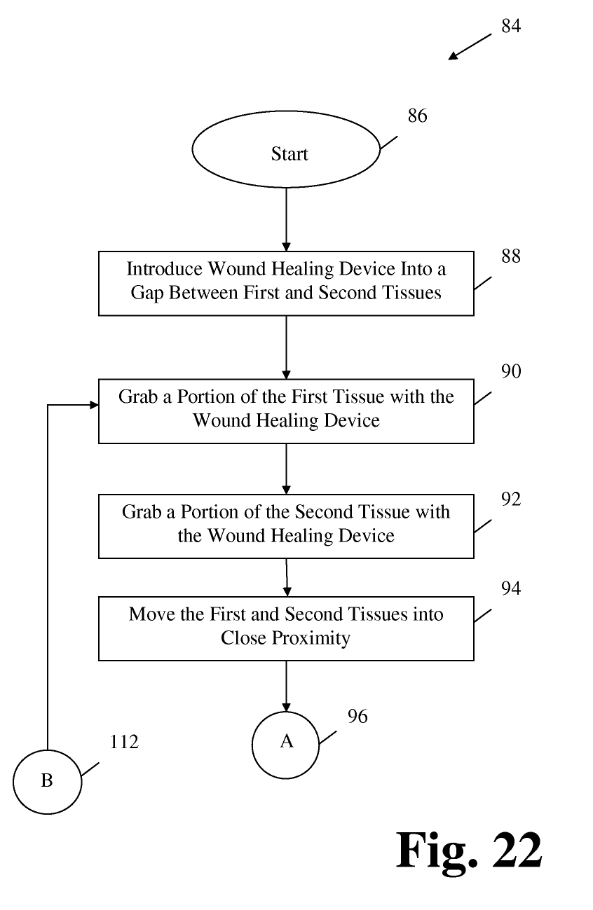

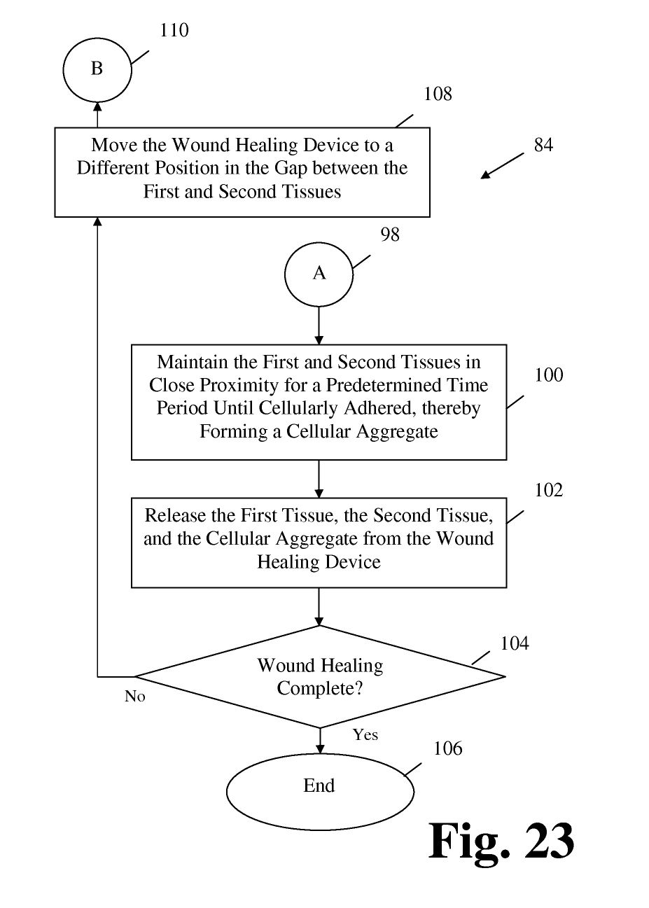

FIGS. 22 and 23 provide a flow chart that summarizes one method contemplated by the present invention.

With reference to FIG. 22, the method 84 begins at 86. In the first operation, the method 84 includes the step of introducing the wound healing device 10, 28 into a gap between the first and second tissue portions 14, 16, which step is designated 88. Then, a portion of the first tissue 14 is grabbed by the wound healing device 10, 28 at 90. A portion of the second tissue 16 is grabbed by the wound healing device 10, 28 at 92. Once acquired, the first and second tissues 14, 16 are moved into close proximity to one another at 94. The method 84 transitions to FIG. 23 at transfer node 96, which is labeled “A.”

With reference to FIG. 23, the method continues from the transfer node “A,” which is labeled 98. At 100, the first and second tissues 14, 16 are maintained in close proximity for a predetermined period of time until the tissues 14, 16 are cellularly adhered. As noted above, the cellular adhesion produces the cellular aggregate 24. Then, at 102, the first tissue 14, the second tissue 16, and the cellular aggregate 24 are released by the wound healing device 10, 28. The method then proceeds to a decision point 104.

At the decision point 104, a determination is made if the wound healing process is complete. If so, the method 84 ends at 106. If the wound healing is not complete, then the method 84 proceeds to step 108 where the wound healing device 10, 28 is moved to a different position in the gap between the first and second tissues 14, 16. From this point, the method returns, via the nodes “B,” labeled 110, 112 to the step 90. The method 84 may then be repeated as needed until the wound is closed.

As should be appreciated from the foregoing discussion, the method 84 is intended to encompass both incremental and continuous wound healing methodologies.

It is noted that the steps identified above and in FIGS. 22 and 23 are not intended to be completely independent steps from one another. Some of the steps may be performed simultaneously or nearly simultaneously with others of the steps. For example, the operations 90, 92 are anticipated to occur nearly simultaneously. Of course, they may also be performed separately without departing from the scope of the invention.

Reference is now made to FIGS. 6-13. It is noted that these illustrations were created using computer-aided drafting (CAD) models, generated using SolidWorks (Dassault Systèmes SolidWorks Corp., Concord, Mass.). These illustrations are intended to be illustrative of the wound healing device 10, 28 and the method 84 that are aspects of the present invention.

Reference is now made to FIGS. 4, 5, and 14-17. As noted above, especially with reference to FIGS. 14-17, these figures are intended to relate how the method 84 may be repeated sequentially within a wound.

Concerning FIGS. 4, 5, and 14-17, among others, and as discussed above, it is contemplated that the wound healing device 10, 28 and the method 84 may incorporate wound “tailoring” or wound “scraping.” Surgeons often aim to “deepen” an incision as they break through successive layers of the skin in a controlled way. The wound healing device 10, 28 may incorporate functionality to shape the wound 12 to facilitate adhesion between the tissues 14, 16.

Intuitively, the wound healing device 10, 28 acts to implement an opposite procedure from wound “tailoring,” in which the wound 12 becomes successively shallower. In order to accommodate the sequence of events needed to effect healing in a three-dimensional wound 12, the tool tip portion 26 contains different elements, each of which implements different actions. As the wound healing device 10, 28 is moved along an open wound 12, features on the front end of the tool 10, 28 prepare the tissue 14, 16 on either side of the wound 12 for re-attachment. A second set of features on the tool 10, 28 are in charge of “zippering” (i.e., re-attaching) cells on either side of the deepest part of the wound 12. After the tool 10, 28 has passed, the deep wound boundary has been brought closer to the surface (i.e., the wound becomes more shallow). Several passes are contemplated with each pass having the tool 10, 28 located at a higher (i.e., more superficial) portion of the wound 12 so that the wound will become shallower after each pass until, after a sufficient number (n) of passes, the wound will be substantially closed.

As discussed above, the tool 10, 28 includes surfaces 46, 48, 50, 52 containing a high density of apertures 56, 58, 60, 62, which function as nozzles though which suction 18, pulsion, and flushing actions (through different holes and independently of each other) are possible.

It is anticipated that gap size may affect performance of the tool 10, 28 and method 84 of the present invention. Gaps ranging from the microscopic (e.g., smaller than 20 microns) to the mesoscopic (e.g., 0.2 millimeters) are expected to be addressable by the tool 10, 28 and the method 84 of the present invention. Other, larger-or smaller-sized wounds 12 also may be addressed by the present invention. Based on pilot data, it is anticipated that, when cells are attracted to the nozzles 56, 58, 60, 62 by suction 18, and then released by pulsion, the cells will stick to one another.

Varying the size and shape of the microscopic nozzles 56, 58, 60, 62 is expected to influence the degree to which cells become deformed during the “suction and release” process. The present invention is intended to encompass a tool 10, 28 that includes apertures 56, 58, 60, 62 of any shape and/or size.

It is contemplated that portions of one or both sides of the tool 10, 28 will be provided with an anti-stick coating, so that the tool 10, 28 may be maneuvered in the wound 12 without causing undue adherence to the sides of the wound 12.

While the tool 10, 28 is contemplated to address dermal wounds, it is contemplated that the tool 10, 28 may be employed regardless of the tissue type (or tissue types). In other words, the tool 10, 28 is not limited to the repair of skin tissue alone.

Anticipated uses include the repair of other soft tissue, alone or in combination, such as muscle, tendons, internal organs, and nerve tissue, as well as temporary or permanent implants. In addition, it is anticipated that the present invention will be used in conjunction with other tools that augment, or are augmented by, the invention. Such tools include visualization systems (e.g., MRI, microscopes, ultra-sound), surgical tools (e.g., scalpel), and substances with pharmacological effects.

The device head 26 may be constructed using a combination of rapid prototyping (e.g., stereo-microlithography, direct metal laser sintering [DMLS]) and microfabrication techniques in order to integrate multi-scale features within the same device.

It is also contemplated that the device 10, 28 may incorporate a digital camera (or equivalent) to inspect the wound 12 during use of the tool 10, 28. Specifically, real-time visualization of the wound 12 may assist with operation of the device 10, 28 and success of the method 84. Alternatively, it is contemplated that the device 10, 28 may not include any type of visualization componentry. Either construction is intended to fall within the scope of the invention.

As should be apparent, with visualization componentry, it may be possible to visualize the entirety of the wound 12 to assist with the formation of the cellular aggregate 24. Alternatively, visualization if a portion of the wound 12 may be equally assistive.

It is anticipated that visualization of the wound 12 may be difficult due to the macroscopic size of the tool 10, 28, which increases the distance between the microscope objective and the wound 12. In order to compensate for this effect, it is contemplated to deploy contact microscopes made of active pixel sensors close to the wound 12. These sensors may be mounted directly upon the tool 10, 28 to monitor therapy in real-time. For example, the first side may include a microscope 150 for observing the tissue and conditions in the gap.

It is noted that modeling concepts have been found useful in many areas of biomedical device development. It is contemplated that, to model the interaction of the device 10, 28 with tissue 14, 16, structural dynamic codes may be used to address the coupled differential equations describing propagation of the zippering closure of the wound 12. (See, e.g., M S Hutson, Y Tokutake, M-S Chiang, J W Bloor, D P Kiehart, G S Edwards, “Forces for Morphogenesis Investigated with Laser Microsurgery and Quantitative Modeling,” Science, Volume 300, pages 145-149 (2003)). Those models relate measurements of attractive forces on the leading edge of the wound to the observed closure velocities. Time-dependent codes are expected to model the dynamic response of viscous fluids to arbitrary nozzle and tool shapes in motion in order to yield predicted forces. It is contemplated to measure the force upon the tool 10, 28, while observing the integrity of the re-attached layers of tissues 14, 16 with a separate force sensor and collecting high resolution optical images. The relationship between predicted and observed measurements may be used to iteratively guide fabrication and improve instructions for use.

In connection with these contemplated embodiments, and as should be apparent from the foregoing discussion, it is contemplated that the device 10, 28 of the present invention may be connected to an automated manipulator that is controlled by a processor, computer, or the like. In this contemplated variation, the tool 10, 28 is guided automatically through the process 84, under the supervision of a qualified surgeon, technician, etc.

Other variations of the tool 10, 28 and method 84 of the present invention are also contemplated.

For example, suction 18 need not be employed solely to draw the tissues 14, 16 together. It is contemplated that dielectrophoresis or electro-osmosis may be employed. Dielectrophoresis relies on electrical fields to assert forces on the tissues 14, 16 of the wound 12. Using this technique, an electrical field may be employed to polarize the cells in the tissues 14, 16. The electrical field may then be used to apply a force on the cells after they have been polarized. Electro-osmosis involves the application of an electrical field to move a fluid, for example. Accordingly, use of this technique may be employed to generate a suction in the wound healing device 10, 28, for example.

Alternatively, mechanical means also may be employed. For example, it is contemplated that the tissue 14, 16 may be grabbed by micro-forceps and brought into close proximity for formation of the cellular aggregate 24. In a continuous process, two side-by-side micro-conveyor belts may be employed to press the tissues 14, 16 together. Of course, any combination of these methodologies, together with suction, also may be employed.

In still another contemplated embodiment, it may be possible to employ various nanotechnologies to assist with closure of the wound 12. For example, it is contemplated that the wound healing device may be provided with a plurality of small pipettes at the tip that attract the tissues 14, 16. The micro-pipettes may then be manipulated via an electrical to magnetic field so that the tissues 14, 16 are brought into close proximity with one another.

As should be apparent from the foregoing, the wound healing device 10, 28 and the method 84 are anticipated to effectuate healing of a wound 12 without the need for traditional surgical closures including sutures, staples, adhesives, or the like. It is also contemplated that the present invention may be employed in combination with these traditional closures to enhance traditional methodologies.

With respect to the amount of time that the tissues 14, 16 are held in close proximity to one another, it is contemplated that a time period of less than about one minute should be sufficient for most tissue types. In many instances, less than about ten seconds should be sufficient to promote cellular aggregation. Further still, the predetermined time period may be less than about 1 second. It is also contemplated that a time period of about 100 milliseconds will be sufficient to engender cellular adhesion.

As should be apparent to those skilled in the art, the amount of time required to promote cellular adhesion depends upon a number of factors including the amount of force 20 applied to the tissue 14, 16. In addition, certain tissues 14, 16 are anticipated to exhibit a higher degree of cellular attraction than other tissue types. Accordingly, while 100 milliseconds may be appropriate for one example of tissue adhesion, another type might require ten seconds or more.

As noted above, there are numerous variations and equivalents of the present invention that should be appreciated by those skilled. The present invention is intended to encompass those equivalents and variations.

What is claimed is:

1. A method for closing a gap between tissues, comprising:
introducing a device head into the gap between a first tissue and a second tissue;
applying a force to a portion of the first tissue with the device head;
applying a force to a portion of the second tissue with the device head;
moving, via the device head, the portion of the first tissue into close proximity to the portion of the second tissue;
maintaining, for a predetermined period of time via the device head, the portion of the first tissue and the portion of the second tissue in close proximity until the portion of the first tissue and the portion of the second tissue become cellularly adhered to one another, thereby forming a cellular aggregate that connects the portion of the first tissue to the portion of the second tissue; and
releasing from the device head the portion of the first tissue, the portion of the second tissue, and the cellular aggregate after the predetermined period of time;
wherein the predetermined portion of time is less than about one minute.

2. The method of claim 1, wherein the predetermined time is less than about ten seconds.

3. The method of claim 1, wherein the predetermined time is less than about 1 second.

4. The method of claim 1, wherein the predetermined time is about 100 milliseconds.

5. The method of claim 1, further comprising:
moving the device head to a different portion of the gap between the first tissue and the second tissue so that a subsequent cellular aggregate forms therebetween.

6. The method of claim 1, wherein the device head is moved continuously from a first end to a second end of the gap between the first tissue and the second tissue to form a continuous cellular aggregate therebetween.

7. The method of claim 1, wherein the formation of the cellular aggregate is followed by the formation of one or more additional cellular aggregates in layers, one being more superficial than another.

8. The method of claim 1, further comprising:
cleaning the gap between the first tissue and the second tissue prior to forming the cellular aggregate.

9. The method of claim 1, further comprising:
surgically altering surfaces of the first tissue and the second tissue to facilitate formation of the cellular aggregate therebetween.

10. An apparatus for closing a gap between tissues, comprising:
a device head for insertion in a gap between a first tissue and a second tissue, wherein the device head comprises
a first side,
a second side region disposed opposite to the first side,
a leading edge, and
a trailing edge disposed opposite to the leading edge;
a first plurality of openings disposed through the first side region;
a second plurality of openings disposed through the second side region; and
a suction generator connected to the first plurality of openings;
wherein application of suction by the suction generator to the first plurality of openings is adapted to pull a portion of the first tissue into close proximity with a portion of the second tissue so that as the device head is moved in the gap the suction permits the portion of the first tissue and the portion of the second tissue to contact and become adhered to one another, thereby forming a cellular aggregate connecting the portion of the first tissue to the portion of the second tissue.

11. The apparatus of claim 10, wherein the suction generator is connected to the second plurality of openings such that suction may be applied to the first plurality of openings and the second plurality of openings to pull a portion of the first tissue into close proximity with a portion of the second tissue so that as the device head is moved in the gap the suction permits the portion of the first tissue and the portion of the second tissue to contact and become adhered to one another, thereby forming a cellular aggregate connecting the portion of the first tissue to the portion of the second tissue.

12. The apparatus of claim 10, further comprising:
a fluid outlet and a fluid inlet disposed adjacent the leading edge; and
a fluid source connected to the fluid outlet to supply fluid thereto;
wherein the fluid inlet is connected to the suction generator such that fluid may be discharged through the fluid outlet for introduction into the gap between the first tissue and the second tissue, and
wherein the fluid in the gap between the first tissue and the second tissue is removed via the fluid inlet.

13. The apparatus of claim 10, wherein the first side and the second side each comprise:
a preparation portion adjacent to the leading edge; and
a healing portion disposed adjacent to the trailing edge,
wherein the preparation portion is adapted to shape the tissue to enhance forming a cellular aggregate.

14. An apparatus adapted for introduction into a wound to interdigitate cells from one side of a wound with cells from another side of the wound.

15. The apparatus of claim 14 wherein the apparatus is adapted to move through the wound.

16. The apparatus of claim 14 further comprising apertures through which suction may be applied.

17. The apparatus of claim 16 further comprising means for applying the suction intermittently.

18. The apparatus of claim 16 wherein the apertures are smaller than one millimeter in diameter

19. A method of wound healing comprising interdigitating cells from one side of a wound with cells from another side of the wound in order to reduce the depth of a wound by intra-cellular adhesion.

20. The method of claim 19 where interdigitating cells occurs incrementally along the length of the wound at a desired depth of the wound and then interdigitating cells is repeated at a different depth along the length of the wound so that the strength of the seam between edges of the wound is increased.

21. The apparatus of claim 10 wherein the first side includes a scalpel to shape the tissue.

22. The apparatus of claim 10 wherein the first side includes means to abrade the tissue.

23. The apparatus of claim 10 wherein the first side includes a microscope for observing the tissue and conditions in the gap.
